(19) United States
(12) Patent Application Publication
(10) Pub. No.: US 2008/0103510 A1
(43) Pub. Date: May 1, 2008

(54) BIO-ABSORBABLE TISSUE CLOSURE SYSTEM

(75) Inventors: Thomas Vincent Taylor, Houston, TX (US); John Edward Parazynski, Bellevue, WA (US); Scott Parazynski, Houston, TX (US)

Correspondence Address: GEORGE R. REARDON, 3356 STATION COURT, LAWRENCVILLE, GA, 30044, US

(73) Assignee: APOGEE SURGICAL INSTRUMENTS, Houston, TX (US)

(21) Appl. No.: 11/923,656

(22) Filed: Oct. 25, 2007

Related U.S. Application Data
(60) Provisional application No. 60/862,970, filed on Oct. 25, 2006.

(51) Int. Cl. A61B 17/064

(52) U.S. Cl. 606/143

(57) ABSTRACT

A surgical fastening system, utilizing surgical clips made from bio-absorbable materials, for use in the rapid closing of deep internal wounds in humans or animals is disclosed. Elements of the system include surgical clips, applicators of surgical clips and dispensers of surgical clips. The surgical clips may contain small amounts of prophylactic antibiotic medication, long-acting time-release pain medication, radio-opaque markers, small amounts of radioactive material, colors, and/or patterns.

This application claims the benefit of the filing date of U.S. Provisional Patent Application 60/862,970 filed on Oct. 25, 2006, which is incorporated herein in its entirety.

### FIELD OF THE INVENTION

The technology disclosed herein relates generally to the field of surgical fastener systems for use in rapid wound closure. The technology disclosed herein relates to more particularly to surgical fastening systems made from polymeric materials for use in the rapid closing of deep internal wounds in humans or animals. Additionally, the technology disclosed herein relates to a bio-absorbable tissue closure system for the closure of adipose and other subcutaneous tissues where dissolvable sutures would otherwise be employed.

### BACKGROUND OF THE INVENTION

When dealing with deep tissue war injuries aggressive forward resuscitative care and stabilization of the trauma patient is critical. This aggressive forward care is coupled with rapid air transport to a facility where more sophisticated surgical techniques may be applied. The time, technique and materials involved for battlefield medical personnel to stop deep tissue bleeding need to have the following characteristics:
The time to stop bleeding needs to be short
The technique needs to simple and require minimal training
The materials need to be readily available, easily dispensed, maintain sterility in the field, and be bio-absorbable; additional desirable characteristics include the ability to:
   be located by the forwarded-to facility, e.g. by x-ray
   to time-release medication, e.g. antibiotics, pain medication

Historically the most prevalent method for forcibly closing a tissue opening is through the use of a suture or stitches. As early as the second century, the Greeks used sutures to physically close skin openings. In its simplest form, a suture is simply a length of material that is attached to a tissue-piercing device, e.g. a needle, and looped through the opposing sides of a tissue opening. The suture is then pulled tight and the loop closes, causing the opposing sides of the tissue opening to come into close physical contact. The suture loop is held tight by the tying of one or more knots, or some other locking mechanism.

The first sutures were made of animal gut. Eventually other natural suture materials including leather, horsehair, flax, cotton and silk came into use. As the sciences of medical and materials technology have advanced over the course of the past century, staples, stapling applicators and bio-absorbable materials have been developed to further improve upon the basic suturing concept.

Surgical fasteners, or staples, are commonly used in surgical procedures to allow a surgeon to fasten body tissue quickly without the need for time-consuming suturing. Such surgical fasteners may be applied by surgical staplers singly, in succession, or a number may be applied simultaneously. The majority of these surgical fasteners are directed to the closure of the top portions of body tissue.

Some types of surgical fasteners are two-part devices. That is, they are composed of a fastener, or staple, portion, which is generally a pronged U-shaped member, and a retainer portion, which has apertures into which the prongs are engaged and held. Such fasteners, once engaged, are not separable. Therefore, once inserted into body tissue they cannot be easily removed. For this reason, two part fasteners generally are made of bio-absorbable material, such as glycolide, lactide, or copolymers of glycolide and lactide.

There are several problems with conventional sutures and staples.

One problem involves the rapid closure of deep traumatic wounds, e.g. in battlefield situations, accidents, terrorist attacks. Present techniques require a skilled surgeon placing numerous stitches, tying each individually. The method requires training, proficiency and is time-consuming.

An additional problem is during surgery in the abdominal cavity of an obese person. In this situation a surgeon often has difficulty suturing or stapling the skin closed because of the thick fat layer just under the skin.

Related art includes the following patents.

U.S. Pat. No. 7,112,214, issued to Peterson et al. on Sep. 26, 2006, discloses a fastener for insertion into pierced openings of a tissue wound has a body formed of a generally bio-absorbable polymer defining an initial capture area internal to the body. The body includes a pair of arms, each with an inwardly projecting cleat operably joined at an elbow portion defining an internal elbow angle. The arms are operably joined to a back-span at a shoulder portion defining an internal shoulder angle. A durable tissue retention zone is defined between the cleat and the arm. The elbow portion and the internal elbow angle define an insertion width greater than a width of the pierced openings resulting in the pierced openings stretching over the cleat and being elastically retained within the durable tissue retention zone. The fastener initially captures wound tissue in the initial capture area and then dynamically reforms in response to lateral stresses applied by the wound tissue without a fracture failure of the fastener until a minimum degradation period.

U.S. Pat. No. 7,028,878 B2, issued to Bauer on Apr. 18, 2006 discloses a mechanical stapling device for fastening deep tissue during the closing of peritoneal side of a stab wound, which is associated with a laparoscopic surgical procedure. Also provided is a unique staple for use with the stapling device of the present invention. A method of using the device and staple of the present invention is also provided.

U.S. Pat. No. 6,867,247, issued to Williams et al. on Mar. 15, 2005 discloses devices formed of or including biocompatible polyhydroxyalkanoates are provided with controlled degradation rates, preferably less than one year under physiological conditions. Preferred devices include sutures, suture fasteners, meniscus repair devices, rivets, tacks, staples, screws (including interference screws), bone plates and bone plating systems, surgical mesh, repair patches, slings, cardiovascular patches, orthopedic pins (including bone filling augmentation material), adhesion barriers, stents, guided tissue repair/regeneration devices, articular cartilage repair devices, nerve guides, tendon repair devices, atrial septal defect repair devices, pericardial patches, bulking and filling agents, vein valves, bone marrow scaffolds, meniscus regeneration devices, ligament and tendon grafts, ocular cell implants, spinal fusion cages, skin substitutes, dural substitutes, bone graft substitutes, bone dowels, wound dressings, and hemostats. The polyhydroxyalkanoates can contain additives, be formed of mixtures of monomers or include pendant groups or modifications in their backbones, or can be chemically modified, all to alter the degradation rates. The polyhydroxyalkanoate compositions also provide favorable mechanical properties, biocompatibility, and degradation times within desirable time frames under physiological conditions.

U.S. Pat. No. 6,551,343, issued to Tormala et al. on Apr. 22, 2003, discloses a surgical fastener or device (implant) formed in the shape of an arrow comprising a shaft with a proximal (first) portion having a blunt end and tapered form, such that the proximal portion is broad at its blunt end and tapers in the direction of a distal (second) portion of the shaft, the distal portion having protrusions and a pointed end, wherein the tapered form of the proximal portion and the protrusions of the distal portion are configured to lock the implant in a position inside a soft and/or tough tissue according to the use of the implant. The implant may be manufactured of a polymer or a polymeric compound which is substantially (bio)absorbable (resorable) after implantation in tissue and contains an oriented reinforcing structure or the like of a polymer or polymeric compound or ceramic bioactive glass compound.

U.S. Pat. No. 6,120,526, issued to Daley on Sep. 19, 2000, discloses bio-absorbable staples, delivery systems for the application of the bio-absorbable staples and methods for tissue closure. The delivery systems include a semi-automatic device for applying one or more bio-absorbable staples and a manually powered device for delivering bio-absorbable staple one at a time.

U.S. Pat. No. 6,090,131, issued to Daley on Jul. 18, 2000, discloses bio-absorbable staples and methods for tissue closure. The first type of staple is a one-piece bio-absorbable staple constructed of an elongate body and a locking mechanism, where the locking mechanism is made up of a retainer having an enclosed central tunnel and is located at the first end of the elongate body, and an arrow head or connector head located at the second end of the elongate body. The second type of staple is a two-piece staple for tissue closure constructed of a first elongate body having a leg connected to an arrow head at each end and a second elongate body having a retainer at each end, where each retainer contains an eyelet. Each method for tissue closure involves grasping and holding the tissue to be closed, forcing a bio-absorbable staple through the tissue, and locking the staple.

U.S. Pat. No. 5,618,311, issued to Gryskiewicz on Apr. 8, 1997, discloses a biodegradable, subcuticular surgical clip having a pair of interconnected arms each having piercing prongs directed toward the other arm. The fastener does not have interlockable members.

U.S. Pat. No. 5,573,541, issued to Green et al. on Nov. 12, 1996, discloses a surgical apparatus for attaching two portions of cutaneous body tissue, includes a pair of opposed elongated members having jaws which have tissue gripping members thereon at one end and which are movable toward each other to a closed position to engage two body tissue portions positioned within the members to move the body tissue portions into close approximation. Manually gripping systems of several alternative configurations are provided to actuate a mechanical transmission system to close the jaws. In a preferred embodiment, a fork is movable from a first position to a second position by manually operable devices to move the jaws toward each other. A pair of cam faces is located adjacent the opposite ends of the jaws. As the fork moves to its second position, the tines of the fork engage against the cam faces to move the opposed jaws to their closed position. A plurality of rod-like fasteners are carried in a stacked configuration proximally of the jaws such that when the jaws are in their closed position and the two body tissue portions are held in close approximation. Each rod-like fastener is movable to a position in ingress to penetrate the body tissue portions to attach the body tissue portions together. In another preferred embodiment, the elongated members are biased outwardly by a coil spring 310 and a unique plate-like fastener drive member is provided to facilitate driving a fastener after the jaws are closed. A fastener cartridge is provided with fasteners stacked in a sloped fashion and having a coil spring 310 to bias the fasteners toward the ejection position. Another embodiment includes a rack and gear teeth arrangement for approximating the jaws and for firing the fasteners. The rack and gear teeth function somewhat like a linear analog of a set of rotary gears. A method of attaching cutaneous body tissue portions is also disclosed.

U.S. Pat. No. 5,489,287, issued to Green et al. on Feb. 6, 1996, discloses a surgical apparatus for attaching adjacent end portions of cutaneous body tissue wherein a pair of opposed jaws have means for moving the jaws toward and away from each other. Body tissue engaging means in the form of sharp tip members extend from each jaw and toward the other so as to engage the opposed portions of cutaneous tissue when the jaws are positioned thereagainst and moved toward each other. The jaws and the tips are arranged to cause the cutaneous tissue to assume an irregular shape at the interface whereby an elongated rod-like member, preferably rectangular in cross section, may be directed generally medially of the interface of the tissue to attach the opposed portions to thereby permit healing. The elongated member may have indentations to improve retention within the tissue. A tapered distal end of the elongated member has a pointed tip to facilitate penetration into the tissue.

U.S. Pat. No. 5,423,856, issued to Green on Jun. 13, 1995, discloses a surgical apparatus for attaching adjacent end portions of cutaneous body tissue wherein a pair of opposed jaws have means for moving the jaws toward and away from each other. Body tissue engaging means in the form of sharp tip members extend from each jaw and toward the other so as to engage the opposed portions of cutaneous tissue when the jaws are positioned there-against and moved toward each other. The jaws and the tips are arranged to cause the cutaneous tissue to assume an irregular shape at the interface whereby an elongated member may be directed generally medially of the interface of the tissue to attach the opposed portions to thereby permit healing. A method of attaching adjacent end portions of cutaneous body tissue surrounding an opening utilizing the apparatus of the invention is also disclosed.

U.S. Pat. No. 5,389,102, issued to Green et al. on Feb. 14, 1995, discloses a surgical apparatus for attaching two portions of cutaneous body tissue comprising a pair of opposed elongated members having jaws which have tissue gripping members thereon at one end and which are movable toward each other to a closed position to engage two body tissue portions positioned within the members to move the body tissue portions into close approximation. Manually gripping systems of several alternative configurations are provided to actuate a mechanical transmission system to close the jaws. In a preferred embodiment, a fork is movable from a first position to a second position by manually operable devices to move the jaws toward each other. A pair of cam faces is located adjacent the opposite ends of the jaws. As the fork moves to its second position, the tines of the fork engage against the cam faces to move the opposed jaws to their closed position. A plurality of rod-like fasteners are carded in a stacked configuration proximally of the jaws such that when the jaws are in their closed position and the two body tissue portions are held in close approximation. Each rod-like fastener is movable to a position in ingress to penetrate the body tissue portions to attach the body tissue portions together. In another preferred embodiment, the elongated members are biased outwardly by a coil spring 310 and a unique plate-like fastener drive member is provided to facilitate driving a fastener after the jaws are closed. A fastener cartridge is provided with fasteners stacked in a sloped fashion and having a coil spring 310 to bias the fasteners toward the ejection position. Another embodiment includes a rack and gear teeth arrangement for approximating the jaws and for firing the fasteners. The rack and gear teeth function somewhat like a linear analog of a set of rotary gears. A method of attaching cutaneous body tissue portions is also disclosed

U.S. Pat. No. 5,293,881, issued to Green et al. on Mar. 15, 1994, discloses a surgical apparatus for attaching two portions of cutaneous body tissue comprising a pair of opposed elongated jaws having members thereon at one end and which are movable toward each other to a closed position to engage two body tissue portions positioned within the members to move the bodily tissue portions into close approximation. Manually gripping systems of several alternative configurations are provided to actuate a mechanical transmission system to close the jaws. In a preferred embodiment, a fork is movable from a first position to a second position by manually operable devices to move the jaws toward each other. A pair of cam faces is located adjacent the opposite ends of the jaws. As the fork moves to its second position, the tines of the fork engage against the cam faces to move the opposed jaws to their closed position. A plurality of rod-like fasteners are carried in a stacked configuration proximally of the jaws such that when the jaws are in their closed position and the two body tissue portions are held in close approximation, each rod-like fastener is movable to a position of ingress to penetrate the body tissue portions to attach the two body tissue portions together. A method of attaching cutaneous body tissue portions is also disclosed.

U.S. Pat. No. 5,292,326, issued to Green et al. on Mar. 8, 1994, discloses a surgical apparatus for attaching two portions of cutaneous body tissue includes a pair of opposed elongated jaws having members thereon at one end and which are movable toward each other to a closed position to engage two body tissue portions positioned within the members to move the bodily tissue portions into close approximation. Manually gripping systems of several alternative configurations are provided to actuate a mechanical transmission system to close the jaws. In a preferred embodiment, a fork is movable from a first position to a second position by manually operable devices to move the jaws toward each other. A pair of cam faces is located adjacent the opposite ends of the jaws. As the fork moves to its second position, the tines of the fork engage against the cam faces to move the opposed jaws to their closed position. A plurality of rod-like fasteners are carried in a stacked configuration proximally of the jaws such that when the jaws are in their closed position and the two body tissue portions are held in close approximation, each rod-like fastener is movable to a position of ingress to penetrate the body tissue portions to attach the two body tissue portions together. A method of attaching cutaneous body tissue portions is also disclosed.

U.S. Pat. No. 4,932,960, issued to Green et al. on Jun. 12, 1990, discloses a bioabsorbable surgical fastener comprising a fastener member and an interlocking retainer member. The retainer member has longitudinally extending slots on the lateral sides of the retainer for allowing transverse expansion of the columnar members into which the prongs of the fastener member are locked. Fins on both the lateral and transverse sides of the prongs provide extra strength, and stability during implantation. Stopping surfaces prevent the fastener's barbed tip from exiting the opposite side of the retainer.

U.S. Pat. No. 4,805,617, issued to Bedi et al. on Feb. 21, 1989, discloses an improved surgical fastener system comprising parallel rows of staples and receivers with the receivers connected to adjacent receivers by a plurality of linkages.

While these patents and other previous methods have attempted to solve the problems that they addressed, none have utilized or disclosed a bio-absorbable tissue closure system for the rapid closure of adipose and other subcutaneous tissues where dissolvable sutures would otherwise be employed, as does embodiments of the technology disclosed herein.

A system that would allow for a minimally trained health professional to safely and reliably close deeper layers in an emergency situation is needed in order to prevent the patient from bleeding to death. A system is also needed to save considerable time for surgeons in an operating room environment.

Therefore, a need exists for a rapid bio-absorbable tissue closure system with these attributes and functionalities. The rapid bio-absorbable tissue closure system according to embodiments of the invention substantially departs from the conventional concepts and designs of the prior art. It can be appreciated that there exists a continuing need for a new and improved rapid bio-absorbable tissue closure system which can be used commercially. In this regard, the technology disclosed herein substantially fulfills these objectives.

The foregoing patent and other information reflect the state of the art of which the inventors are aware and are tendered with a view toward discharging the inventors' acknowledged duty of candor in disclosing information that may be pertinent to the patentability of the technology disclosed herein. It is respectfully stipulated, however, that the foregoing patent and other information do not teach or render obvious, singly or when considered in combination, the inventors' claimed invention.

### BRIEF SUMMARY OF THE INVENTION

The general purpose of the technology disclosed herein, which will be described subsequently in greater detail, is to provide a bio-absorbable tissue closure system. In particular, the technology disclosed herein relates to a bio-absorbable tissue closure system for the rapid closure of adipose and other subcutaneous tissues where dissolvable sutures would otherwise be employed.

The technology disclosed herein allows the adipose tissue to be drawn together and closed with a simple-to-operate surgical clip applicator, e.g. a stapler-like device, in advance of the closure of the skin. While metallic skin staples require removal by a trained health professional, subcutaneous surgical clips are made of bio-absorbable material that requires no further attention.

The technology disclosed herein also is directed to the use of a surgical clip and an applicator to facilitate the closure of fat, e.g. on either side of an incision in advance of the cutaneous closure at the conclusion of a surgical procedure.

In one embodiment, the distal ends of the applicator are pointed and each lever has an anvil to hold an open surgical clip in place near the distal ends. The points of the applicator tips allow the ends of the open surgical clip to be inserted into the fat tissue, one point on either side of the opening. The applicator effects closure of the surgical clip by pressing the proximal handles together, but there is relatively little movement of the tips of the applicator towards closure; the surgical clip is instead curved against one or both of the anvils into the closed position, preferably in the form of an open loop. The applicator can then be opened to release the surgical clip and retracted from the subcutaneous tissue. The closed surgical clip holds the subcutaneous tissue in place to close the incision, and greatly facilitates the closure of the cutaneous layer over the fat layer.

The technology disclosed herein is also directed to a surgical clip comprised of an elongated body of resorbable or biologically inert material having at least one sharp end in spaced-apart relation to a catch end, where the sharp end has a hook for engaging the catch end, and a curved section between the sharp end and the catch end for bending the body into a loop, so that the hook engages the catch end. The catch end may be further comprised of an eyelet for receiving the hook. Alternatively the catch end may be comprised of a hook opposing the hook of the sharp end for inter-engagement of the opposing hooks in an offset configuration.

The technology disclosed herein also is directed to an applicator for closing subcutaneous tissue using the surgical clip just described, where the applicator is comprised of proximal handle levers, a fulcrum, distal ends tapered to a point, and anvils for receiving the surgical clip in opposing surfaces of the distal ends, where at least one of the distal ends is profiled with the respective opposing surface following the sharp end of the surgical clip, where the anvils are adapted for holding a surgical clip in an open position and where at least one of the anvils is adapted to bend the surgical clip into a closed position to form a loop.

The technology disclosed herein is also directed to a method for closing subcutaneous tissue with the applicator of the preceding paragraph, where the method comprises:
loading a surgical clip into an applicator with the catch end against one of the anvils and the curved section against the other of the anvils
inserting the distal ends of the applicator into the tissue to be closed on either side of an incision
pressing the proximal handle levers together to engage the hook in the catch end to form the surgical clip into a closed loop, thus securing the tissue
releasing the closed loop from the applicator, and
retracting the distal ends of the applicator from the tissue

In one embodiment the bio-absorbable surgical clip has sharp, serrated distal ends operable for insertion directly into tissue and the applicator may bend the surgical clip so that the tips interlock securely thus leaving no sharp surfaces exposed once the surgical clip is closed.

The applicator anvils and horns engage the surgical clip, thus enabling retention of the surgical clip during handling and also allowing for opening and closing of the surgical clip.

In another embodiment of the applicator the handles are rotated approximately 90 degrees to the surgical plane which allows a more ergonomic orientation and provides an improved mechanical advantage to the surgeon.

In at least one embodiment the applicator is operable for a surgeon to adjust the angle of application, e.g. adjustability of the angle between the proximal handles and the surgical clip, allowing the surgeon to adjust the angle of attack based on tissue depth (not shown).

The surgical clip may be formed such that the amount, thickness or composition of the surgical clip determines the rate of resorption by the body, e.g., for surgical repairs requiring structural integrity for longer periods of time a longer lasting surgical clip may be needed.

The surgical clip may have the bio-absorbable material formed in one or more colors for easier viewing within the surgical wound.

The surgical clip may have radio-opaque markers embedded within the surgical clip to aid in postoperative visualization and/or for directing radiation therapy in cancer care.

The surgical clip may incorporate small amounts of radioactive material for targeted postoperative radiation therapy in cancer care.

The surgical clip may have small amounts of prophylactic antibiotic medication embedded in the surgical clip, as well as long-acting, time-release pain medication.

The surgical clip and applicator may be constructed to be applied to other tissue types, e.g., tendons, dura (covering of the brain) and muscle, e.g., an enveloping sheath might be used for tendon repairs since tendon repairs are often difficult to repair as the suturing often munches/reduces the length of the remaining tendon. Having a smooth, resorbable material aids recovery.

An embodiment of the surgical clip is operable for rapid closure of cardiac muscle during heart surgery or penetrating trauma resuscitation. A disk with barbed spikes, fabricated from bio-absorbable material, is inserted into the cardiac chamber and pulled outward; a matching, fenestrated disk clamshells the heart muscle, with the barbs pulling through the fenestrations, thus sealing the heart tissue.

An embodiment of the applicator may interface with a dispenser holding a magazine of a quantity of surgical clips. A magazine of surgical clips may be quickly inserted into the dispenser without breaking the sterile field. This dispenser dispenses surgical clips into the applicator, and subsequently the patient, at a faster rate than manual loading and dispensing.

In one embodiment of the dispenser the surgical clips are advanced by spring force or other mechanical force into an anvil or other feature of the applicator after the preceding surgical clip has been dispensed into the surgical site.

In one embodiment of the dispenser surgical clips of various sizes are accepted, as dictated by the needs of the surgical repair.

One use of the assembly of the applicator, the dispenser and the magazine of surgical clips is on human patients; another use is on veterinary patients.

In one embodiment of the technology disclosed herein the means to package and rapidly dispense multiple surgical clips from a magazine and the applicator tool into a surgical wound is as follows:
Substantially flat surgical clips operable for being stacked one on top of the other are packaged in a magazine holding at least 10 surgical clips in a very compact space
Characteristics of the magazine are as follows:
   Has transparent or partially transparent walls to enable viewing of the surgical clips remaining inside
   As the surgical clips are dispensed, a visible, e.g. opaque/brightly colored, line will advance along the side of the magazine, quickly indicating to the surgeon roughly how many surgical clips remain inside
   A light force, e.g. spring, is imparted from the top of the stack of surgical clips towards the applicator entry point
   As one surgical clip is inserted into the patient, the jaws of the applicator are free to receive another surgical clip;
   the action of closing a surgical clip in the patient and relaxing the handles of the applicator (aided in the opening direction with another force, e.g. a spring) enables yet another spring, or equivalent, to push the next surgical clip, e.g., via a plunger 320, into the applicator jaws
   Grooves in the applicator jaws may guide the surgical clips from the bottom of the magazine (above the top plane of the applicator jaws), down to engage the horns

In another embodiment of the technology disclosed herein the means to package and rapidly dispense multiple surgical clips from a magazine and the applicator tool into a surgical wound is as follows:
A magazine is installed substantially flush to the applicator handles with the surgical clips stacked front-to-back, either because of surgical clip irregular height or to optimize visibility while handling the tool
Characteristics of the magazine are as follows:
   Long, thin, and constructed of transparent walls to enable viewing of the surgical clips remaining inside
   A easy to view indicator strip that becomes more and more visible as surgical clips are dispensed
   A light spring force along the axis of the magazine and applicator pushes the surgical clips forward via a plunger-like element directly into the applicator jaws
   As one surgical clip is dispensed this frees up the jaws to receive another surgical clip
   Grooves in the applicator jaws guides each surgical clip slightly downward and into the applicator tip, engaging the prominent features of the surgical clip, e.g., horns

Additional elements that may be included in embodiments of the technology disclosed herein include:
A central pointer built into the very front of either the magazine or applicator showing where the midline of the closed surgical wound will be once the surgical clip is closed; this will aid the surgeon in assessing alignment as well as depth of surgical clip penetration
Magazines operable to be clicked into place using a small frame and ball-detent device on top of the applicator; a matching hole on the magazine will match fit these features
Magazines that are replaceable without breaking sterility
An applicator design that accommodates more than one size of surgical clip, where the magazine outer dimensions are held substantially constant
Magazines that are clearly labeled for size with an indication on their use
Alternate embodiment: applicator handles may be positioned perpendicular to the jaws, much like a conventional stapler, to facilitate surgical clip packaging and reloading
Optional ability to recycle the magazines (refill, sterilize, re-use)
Outer mold line of magazine is knurled or has features that aid in easy removal and reinstallation into the applicator
Magazine has a radio-opaque marker embedded within, such that if an empty magazine were unintentionally left in a patient it could be quickly found using conventional radiological techniques
An LED (light emitting diode) is built into the magazine on the side facing the outflow (towards the surgical site); as the magazine is introduced into the applicator, a circuit is completed, thus illuminating the surgical site; this may be accomplished via a small coin battery and LED, e.g. a white LED, built into the magazine housing; this facilitates closure when working deep in adipose layers, or in a bloody surgical wound, or in conditions where there is limited illumination.

One aspect of the technology disclosed herein is that it facilitates rapid closure of deep tissue wounds.

Another aspect of the technology disclosed herein is that it facilitates an increased survival rate of deep tissue wound victims in danger of bleeding to death.

Another aspect of the technology disclosed herein is that it is easy to use.

Another aspect of the technology disclosed herein is that it requires minimal training.

Another aspect of the technology disclosed herein is that the surgical clip dispensing system is substantially sterile.

Another aspect of the technology disclosed herein is that it may be made from readily available materials.

Another aspect of the technology disclosed herein is that it may be economically produced.

These and other features and advantages of the technology disclosed herein will be presented in more detail in the following specification of the invention and the accompanying figures, which illustrate by way of example the principles of the invention.

There are additional features of the invention that will be described hereinafter and which will form the subject matter of the claims appended hereto. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the technology disclosed herein. It is important, therefore, that the claims be regarded as including such equivalent constructions insofar as they do not depart from the spirit and scope of the technology disclosed herein.

### BRIEF DESCRIPTION OF THE SEVERAL VIEWS OF THE DRAWINGS

The invention, together with further advantages thereof, may best be understood by reference to the following description taken in conjunction with the accompanying drawings in which:

FIG. 1 illustrates an exploded view of a bio-absorbable tissue closure system, according to an embodiment of the technology disclosed herein.

FIG. 2 illustrates an exploded view of a bio-absorbable tissue closure system, according to an embodiment of the technology disclosed herein.

FIG. 3 illustrates a perspective view of bio-absorbable tissue closure system showing the cooperation between the applicator the dispenser and the clips to close a wound in flesh, according to an embodiment of the technology disclosed herein.

FIG. 4 illustrates a top plan view of a bio-absorbable tissue closure system showing a plunger for advancement of the clips, according to an embodiment of the technology disclosed herein.

FIG. 5 illustrates a top plan view of a bio-absorbable tissue closure system showing a spring for advancement of the clips, according to an embodiment of the technology disclosed herein.

FIG. 6 illustrates a top plan view of a bio-absorbable tissue closure system showing an open clip between the anvils of the applicator, according to an embodiment of the technology disclosed herein.

FIG. 7 illustrates a top plan view of a bio-absorbable tissue closure system showing a closed clip between the anvils of the applicator, according to an embodiment of the technology disclosed herein.

FIG. 8 illustrates a right side plan view of a bio-absorbable tissue closure system, according to an embodiment of the technology disclosed herein.

FIG. 9A illustrates a top plan view of an open surgical clip, according to an embodiment of the technology disclosed herein.

FIG. 9B illustrates a top plan view of a closed surgical clip, according to an embodiment of the technology disclosed herein.

FIG. 9C illustrates a top plan view of a magazine of surgical clips, according to an embodiment of the technology disclosed herein.

FIG. 10 illustrates radio-opaque surgical clips in a simulated x-ray, according to an embodiment of the technology disclosed herein.

### DETAILED DESCRIPTION OF THE INVENTION

The technology disclosed herein will now be described in detail with reference to a few preferred embodiments thereof as illustrated in the accompanying drawings. In the following description, numerous specific details are set forth in order to provide a thorough understanding of the technology disclosed herein. It will be apparent, however, to one skilled in the art, that the technology disclosed herein may be practiced without some or all of these specific details. In other instances, well known operations have not been described in detail so not to unnecessarily obscure the technology disclosed herein.

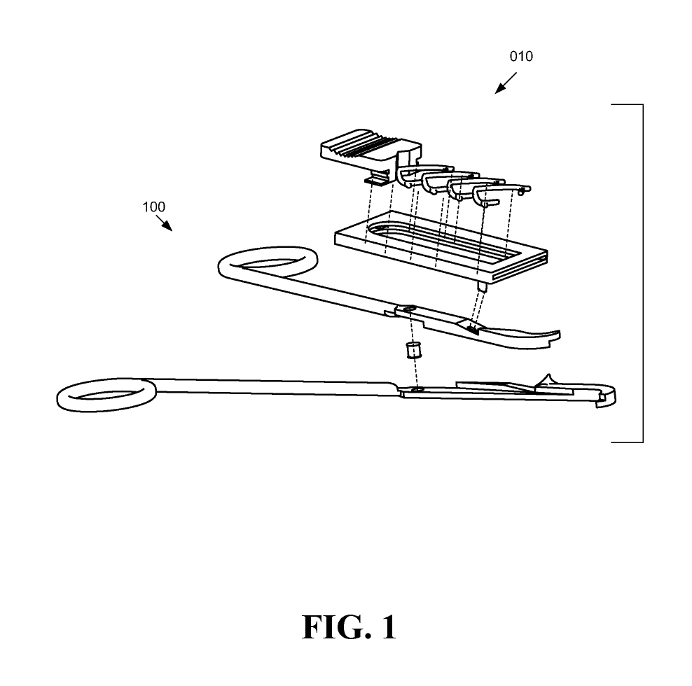

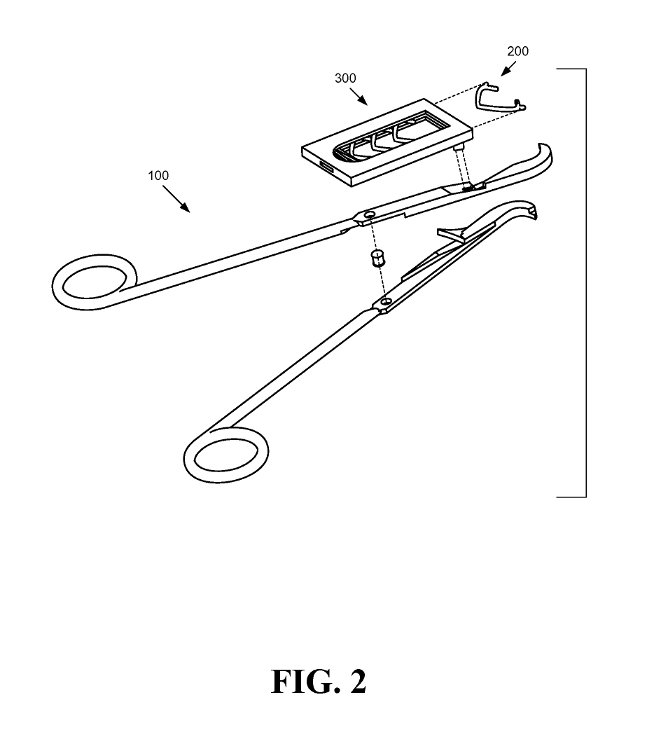

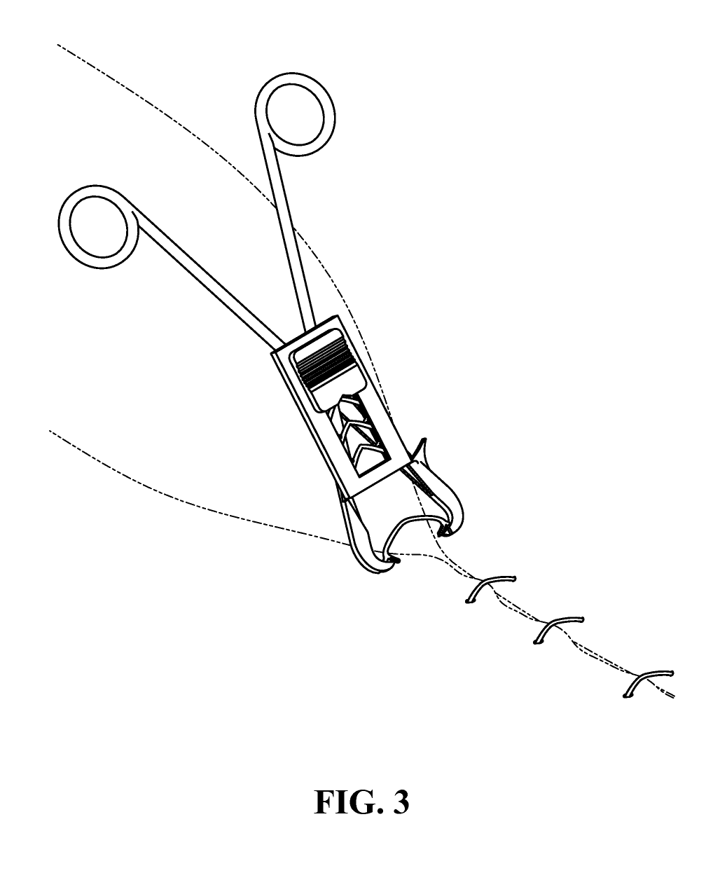

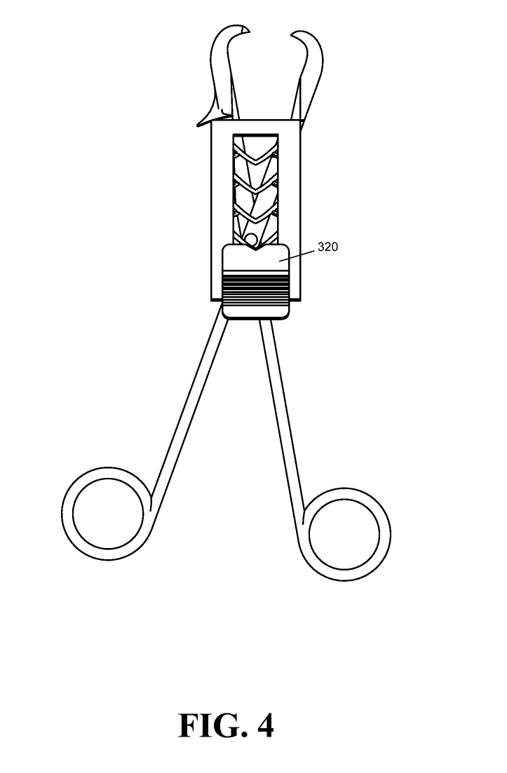

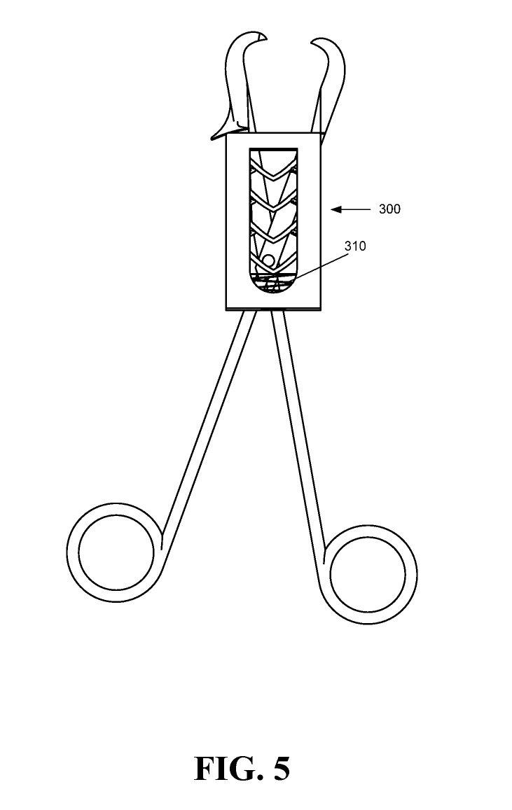

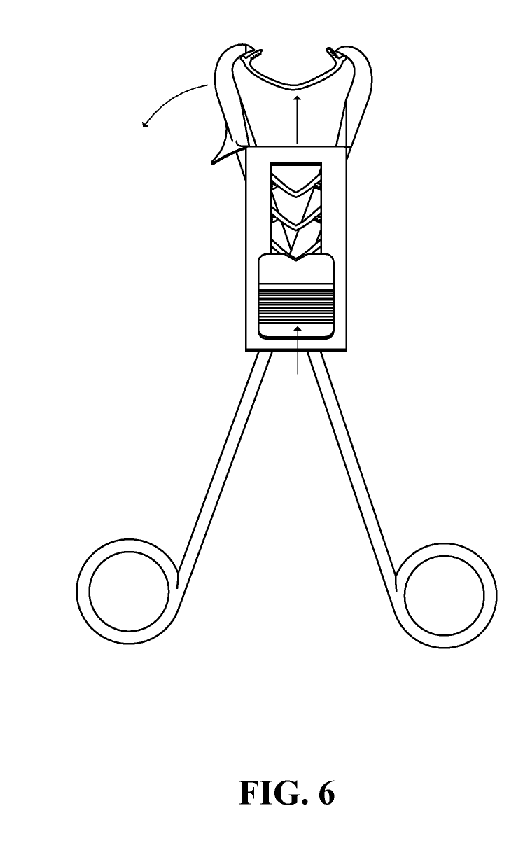

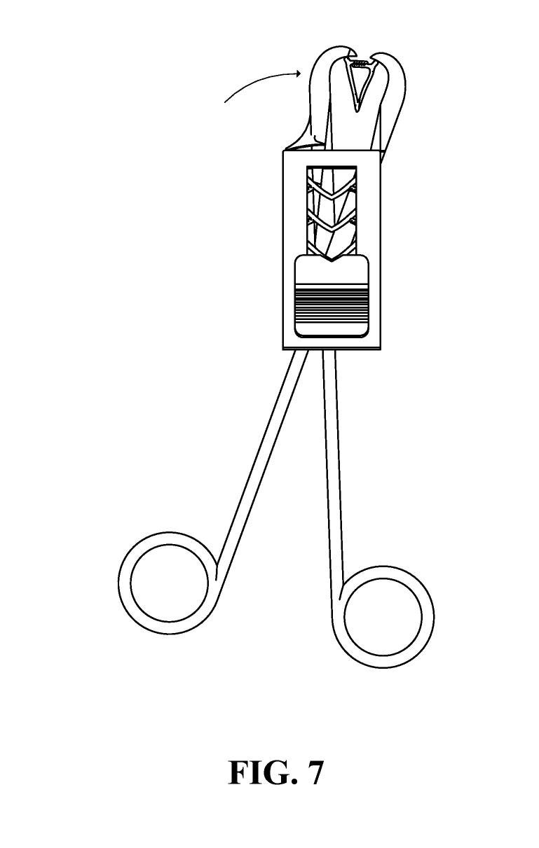

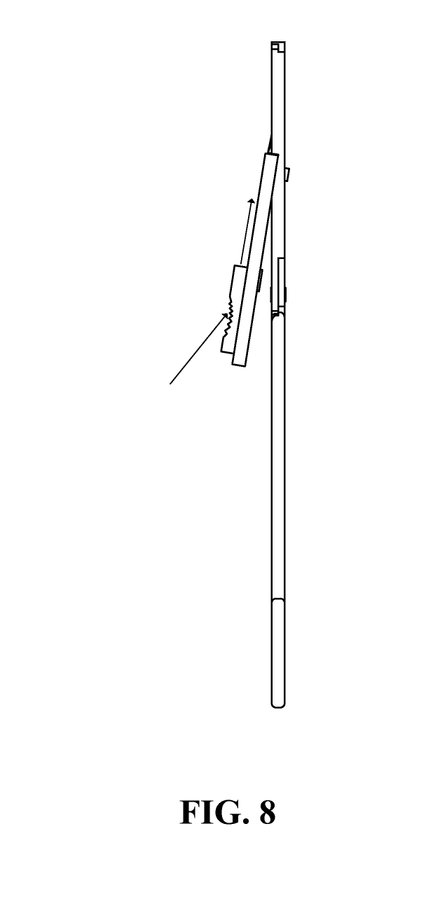

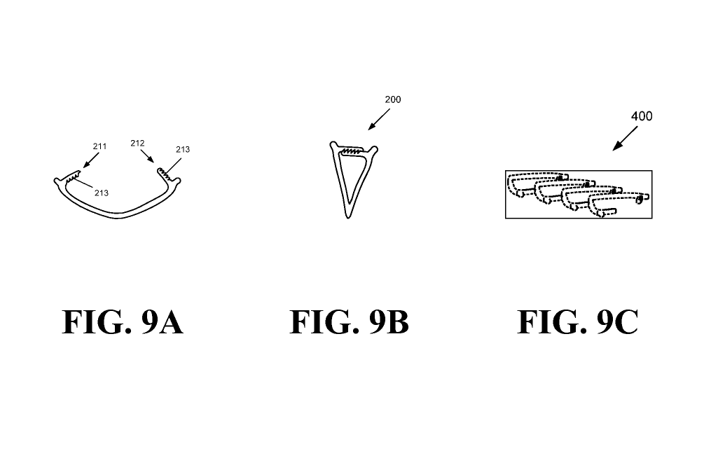

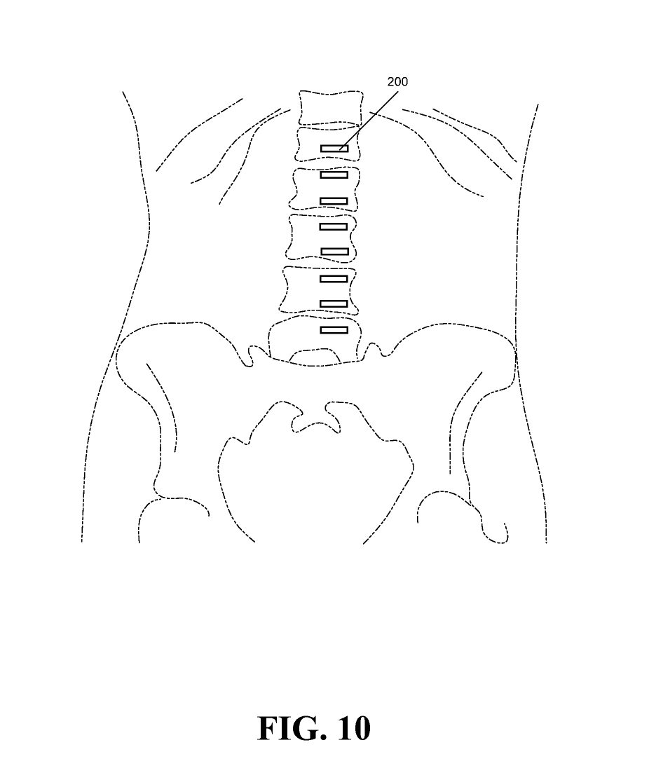

Referring now to FIG. 1 through FIG. 10 an embodiment of a tissue closure clip dispenser system 010 is comprised of an applicator 100 for a bio-absorbable surgical clip 200. The tissue closure clip dispenser system 010 is further comprised of a dispenser 300 into which a magazine 400 containing a plurality of bio-absorbable surgical clips 200 is placed. The dispenser 300 attaches to the applicator 100 and delivers bio-absorbable surgical clips 200 to the tissue engaging elements of the applicator 100.

The technology disclosed herein and methods thereof are also described in the following numbered items:

1. A tissue closure clip dispenser system 010 to dispense, bend, insert, and lock a plurality of bio-absorbable surgical clips 200 for the rapid closure of a deep tissue wound, the system comprising:
a plurality of bio-absorbable surgical clips 200, each bio-absorbable surgical clip 200 formed as a unitary structure having a flexible elongated body with a first distal end 211 and a second distal end 212, where the first distal end 211 and the second distal end 212 comprise a means for interlocking;
a magazine 400 configured to contain the plurality of bio-absorbable surgical clips 200 before any of the plurality of bio-absorbable surgical clips 200 is dispensed in a sterile field;
a dispenser 300 into which the magazine 400 of the plurality of bio-absorbable surgical clips 200 is loaded without breaking the sterile field, the dispenser 300 configured to rapidly dispense the plurality of bio-absorbable surgical clips 200; and
an applicator to receive a dispensed bio-absorbable surgical clip from the dispenser 300, to rapidly bend, insert, and lock the dispensed bio-absorbable surgical clip 200 into subcutaneous tissue, and to thereafter automatically receive a next dispensed bio-absorbable surgical clip 200 from the dispenser 300.
   2. The tissue closure clip dispenser system 010 to dispense, bend, insert, and lock a plurality of bio-absorbable surgical clips 200 for the rapid closure of a deep tissue wound of Item 1, further comprising:
a means to mechanically advance each of the plurality of bio-absorbable surgical clips 200 from the magazine 400 through the dispenser 300 as a preceding bio-absorbable surgical clip 200 has been dispensed through the dispenser 300.
   3. The tissue closure clip dispenser system 010 to dispense, bend, insert, and lock a plurality of bio-absorbable surgical clips 200 for the rapid closure of a deep tissue wound of Item 2, where the means to mechanically advance each of the plurality of bio-absorbable surgical clips 200 comprises a spring 310 and plunger 320 to mechanically and rapidly advance the plurality of bio-absorbable surgical clips 200 each time one of the plurality of bio-absorbable surgical clips 200 is dispensed from the applicator.
   4. The tissue closure clip dispenser system 010 to dispense, bend, insert, and lock a plurality of bio-absorbable surgical clips 200 for the rapid closure of a deep tissue wound of Item 1, where the dispenser 300 is configured to accept a plurality of sizes of bio-absorbable surgical clips 200 to accommodate varying medical needs in subcutaneous tissue repair.
   5. The tissue closure clip dispenser system 010 to dispense, bend, insert, and lock a plurality of bio-absorbable surgical clips 200 for the rapid closure of a deep tissue wound of Item 1, where the magazine 400 is configured to be at least partially transparent and to have a conspicuous line of advancement, providing a means for indicating the number of bio-absorbable surgical clips 200 remaining in the magazine 400.
   6. The tissue closure clip dispenser system 010 to dispense, bend, insert, and lock a plurality of bio-absorbable surgical clips 200 for the rapid closure of a deep tissue wound of Item 1, where the first distal end 211 and the second distal end 212 of each of the plurality of bio-absorbable surgical clips 200 further comprises a plurality of serrations 213, thereby providing the means for interlocking each of the plurality of bio-absorbable surgical clips 200 as the first distal end 211 is engaged with the second distal end 212 once each of the plurality of bio-absorbable surgical clips 200 is inserted into subcutaneous tissue and locked by the applicator.
   7. The tissue closure clip dispenser system 010 to dispense, bend, insert, and lock a plurality of bio-absorbable surgical clips 200 for the rapid closure of a deep tissue wound of Item 1, where the first distal end 211 of each of the plurality of bio-absorbable surgical clips 200 further comprises a sharp end with a hook, and where the second distal end 212 of each of the plurality of bio-absorbable surgical clips 200 further comprises a catch end to receive the sharp end with the hook as the applicator locks each of the plurality of bio-absorbable surgical clips 200 advanced between the sharp end with the hook and the catch end.
   8. The tissue closure clip dispenser system 010 to dispense, bend, insert, and lock a plurality of bio-absorbable surgical clips 200 for the rapid closure of a deep tissue wound of Item 1, where the applicator to receive a dispensed bio-absorbable surgical clip 200 from the dispenser 300 further comprises:
a plurality of proximal handle levers to be held by an operator to operate the tissue closure clip dispenser system 010;
a fulcrum about which the proximal handle levers pivot while in use by the operator dispensing the plurality of bio-absorbable surgical clips 200; and
a set of distal ends having anvils to receive and hold each of the plurality of bio-absorbable surgical clips 200 from the magazine 400 and the dispenser 300 in opposing surfaces of the distal ends and to subsequently bend each of the plurality of bio-absorbable surgical clips 200 into a closed position.
   9. The tissue closure clip dispenser system 010 to dispense, bend, insert, and lock a plurality of bio-absorbable surgical clips 200 for the rapid closure of a deep tissue wound of Item 1, where each of the plurality of bio-absorbable surgical clips 200 comprises more than one color to aid viewing in a surgical repair.
   10. The tissue closure clip dispenser system 010 to dispense, bend, insert, and lock a plurality of bio-absorbable surgical clips 200 for the rapid closure of a deep tissue wound of Item 1, where the plurality of bio-absorbable surgical clips 200 are configured and utilized to facilitate the closure of fat on either side of an incision in advance of a cutaneous closure.
   11. The tissue closure clip dispenser system 010 to dispense, bend, insert, and lock a plurality of bio-absorbable surgical clips 200 for the rapid closure of a deep tissue wound of Item 1, where each of the plurality of bio-absorbable surgical clips 200 further comprises radio-opaque markers embedded within to aid in postoperative visualization and radiation therapy.
   12. The tissue closure clip dispenser system 010 to dispense, bend, insert, and lock a plurality of bio-absorbable surgical clips 200 for the rapid closure of a deep tissue wound of Item 1, where each of the plurality of bio-absorbable surgical clips 200 further comprises antibiotic and time-release pain medication embedded within.
   13. The tissue closure clip dispenser system 010 to dispense, bend, insert, and lock a plurality of bio-absorbable surgical clips 200 for the rapid closure of a deep tissue wound of Item 8, where the set of distal ends having anvils to receive and hold each of the plurality of bio-absorbable surgical clips 200 from the magazine 400 and the dispenser 300 in opposing surfaces of the distal ends and to subsequently bend each of the plurality of bio-absorbable surgical clips 200 into a closed position is further comprised of grooves to guide each of the plurality of bio-absorbable surgical clips 200 into tissue.
   14. The tissue closure clip dispenser system 010 to dispense, bend, insert, and lock a plurality of bio-absorbable surgical clips 200 for the rapid closure of a deep tissue wound of Item 1, further comprising:
a central pointer, integrally formed in the magazine 400 or the applicator 100 of the tissue closure clip dispenser system 010, highlighting where the midline of the closed surgical wound will be once each of the plurality of bio-absorbable surgical clips 200 is closed, thereby aiding a surgeon in assessing alignment and depth of surgical clip penetration.
   15. The tissue closure clip dispenser system 010 to dispense, bend, insert, and lock a plurality of bio-absorbable surgical clips 200 for the rapid closure of a deep tissue wound of Item 1, where the magazine 400 further comprises a radio-opaque marker embedded within.
   16. The tissue closure clip dispenser system 010 to dispense, bend, insert, and lock a plurality of bio-absorbable surgical clips 200 for the rapid closure of a deep tissue wound of Item 1, where the magazine 400 further comprises a light-emitting diode (LED) locating at an end of the magazine 400 facing a surgical site, thereby illuminating the surgical site.
   17. The tissue closure clip dispenser system 010 to dispense, bend, insert, and lock a plurality of bio-absorbable surgical clips 200 for the rapid closure of a deep tissue wound of Item 1, where each of the plurality of bio-absorbable surgical clips 200 is comprised of one or more colors for conspicuous viewing during a tissue repair procedure.
   18. The tissue closure clip dispenser system 010 to dispense, bend, insert, and lock a plurality of bio-absorbable surgical clips 200 for the rapid closure of a deep tissue wound of Item 1, where each of the plurality of bio-absorbable surgical clips 200 is configured for a predetermined rate of resorption within a body, thereby accommodating surgical repairs requiring varied levels of structural integrity and longer periods of recuperation.
   19. A method for loading a plurality of bio-absorbable surgical clips 200 into a tissue closure clip dispenser system 010, the method comprising:
configuring a tissue closure clip dispenser system 010 to comprise:
   a plurality of bio-absorbable surgical clips 200, each bio-absorbable surgical clip formed as a unitary structure having a flexible elongated body with a first distal end 211 and a second distal end 212, where the first distal end 211 and the second distal end 212 comprise a means for interlocking;
   a magazine 400 configured to contain the plurality of bio-absorbable surgical clips 200 before any of the plurality of bio-absorbable surgical clips 200 is dispensed in a sterile field;
   a dispenser 300 into which the magazine 400 of the plurality of bio-absorbable surgical clips 200 is loaded without breaking the sterile field, the dispenser 300 configured to rapidly dispense the plurality of bio-absorbable surgical clips 200; and
   an applicator 100 to receive a dispensed bio-absorbable surgical clip from the dispenser 300, to rapidly bend, insert, and lock the dispensed bio-absorbable surgical clip into subcutaneous tissue, and to thereafter automatically receive a next dispensed bio-absorbable surgical clip from the dispenser 300;
loading the plurality of bio-absorbable surgical clips 200 into the magazine 400;
placing the magazine 400 of the plurality of bio-absorbable surgical clips 200 into the dispenser 300;
installing the dispenser 300 containing the magazine 400 of the plurality of bio-absorbable surgical clips 200, in a substantially flush manner, to the applicator 100; and
replacing the magazine 400 of the plurality of bio-absorbable surgical clips 200 as needed during a closure of a deep tissue wound.
   20. A method for rapidly dispensing a plurality of bio-absorbable surgical clips 200 into subcutaneous tissue and for rapidly closing a deep tissue wound, the method comprising:
configuring a tissue closure clip dispenser system 010 to comprise:
   a plurality of bio-absorbable surgical clips 200, each bio-absorbable surgical clip formed as a unitary structure having a flexible elongated body with a first distal end 211 and a second distal end 212, where the first distal end 211 and the second distal end 212 comprise a means for interlocking;
   a magazine 400 configured to contain the plurality of bio-absorbable surgical clips 200 before any of the plurality of bio-absorbable surgical clips 200 is dispensed in a sterile field;
   a dispenser 300 into which the magazine 400 of the plurality of bio-absorbable surgical clips 200 is loaded without breaking the sterile field, the dispenser 300 configured to rapidly dispense the plurality of bio-absorbable surgical clips 200; and
   an applicator 100 comprising a plurality of proximal handle levers, a fulcrum, and a set of distal ends having anvils, the applicator 100 to receive a dispensed bio-absorbable surgical clip from the dispenser 300, to rapidly bend, insert, and lock the dispensed bio-absorbable surgical clip into subcutaneous tissue, and to thereafter automatically receive a next dispensed bio-absorbable surgical clip from the dispenser 300;
loading the tissue closure clip dispenser system 010 with the plurality of bio-absorbable surgical clips 200;
inserting the distal ends of the applicator 100 into a tissue area to be closed;
pressing the proximal handle levers together to engage one of the plurality of bio-absorbable surgical clips 200 into a closed position;
releasing the closed bio-absorbable surgical clip from the applicator 100;
dispensing additional bio-absorbable surgical clips 200 as required by the tissue area being treated; and
retracting the distal ends of the applicator 100 from the tissue area.

The foregoing description and drawings comprise illustrative embodiments of the technology disclosed herein. Having thus described exemplary embodiments of the technology disclosed herein, it should be noted by those skilled in the art that the within disclosures are exemplary only, and that various other alternatives, adaptations and modifications may be made within the scope of the technology disclosed herein. Merely listing or numbering the steps of a method in a certain order does not constitute any limitation on the order of the steps of that method. Many modifications and other embodiments of the invention will come to mind to one skilled in the art to which this invention pertains having the benefit of the teachings presented in the foregoing description and the associated drawings. Although specific terms may be employed herein, they are used in a generic and descriptive sense only and not for purposes of limitation. Accordingly, the technology disclosed herein is not limited to the specific embodiments illustrated herein, but is limited only by the following claims.

What is claimed is:

1. A tissue closure clip dispenser system to dispense, bend, insert, and lock a plurality of bio-absorbable surgical clips for the rapid closure of a deep tissue wound, the system comprising:
a plurality of bio-absorbable surgical clips, each bio-absorbable surgical clip formed as a unitary structure having a flexible elongated body with a first distal end and a second distal end, wherein the first distal end and the second distal end comprise a means for interlocking;
a magazine configured to contain the plurality of bio-absorbable surgical clips before any of the plurality of bio-absorbable surgical clips is dispensed in a sterile field;
a dispenser into which the magazine of the plurality of bio-absorbable surgical clips is loaded without breaking the sterile field, the dispenser configured to rapidly dispense the plurality of bio-absorbable surgical clips; and
an applicator to receive a dispensed bio-absorbable surgical clip from the dispenser, to rapidly bend, insert, and lock the dispensed bio-absorbable surgical clip into subcutaneous tissue, and to thereafter automatically receive a next dispensed bio-absorbable surgical clip from the dispenser.

2. The tissue closure clip dispenser system to dispense, bend, insert, and lock a plurality of bio-absorbable surgical clips for the rapid closure of a deep tissue wound of claim 1, further comprising:
a means to mechanically advance each of the plurality of bio-absorbable surgical clips from the magazine through the dispenser as a preceding bio-absorbable surgical clip has been dispensed through the dispenser.

3. The tissue closure clip dispenser system to dispense, bend, insert, and lock a plurality of bio-absorbable surgical clips for the rapid closure of a deep tissue wound of claim 2, wherein the means to mechanically advance each of the plurality of bio-absorbable surgical clips comprises a spring and plunger to mechanically and rapidly advance the plurality of bio-absorbable surgical clips each time one of the plurality of bio-absorbable surgical clips is dispensed from the applicator.

4. The tissue closure clip dispenser system to dispense, bend, insert, and lock a plurality of bio-absorbable surgical clips for the rapid closure of a deep tissue wound of claim 1, wherein the dispenser is configured to accept a plurality of sizes of bio-absorbable surgical clips to accommodate varying medical needs in subcutaneous tissue repair.

5. The tissue closure clip dispenser system to dispense, bend, insert, and lock a plurality of bio-absorbable surgical clips for the rapid closure of a deep tissue wound of claim 1, wherein the magazine is configured to be at least partially transparent and to have a conspicuous line of advancement, providing a means for indicating the number of bio-absorbable surgical clips remaining in the magazine.

6. The tissue closure clip dispenser system to dispense, bend, insert, and lock a plurality of bio-absorbable surgical clips for the rapid closure of a deep tissue wound of claim 1, wherein the first distal end and the second distal end of each of the plurality of bio-absorbable surgical clips further comprises a plurality of serrations, thereby providing the means for interlocking each of the plurality of bio-absorbable surgical clips as the first distal end is engaged with the second distal end once each of the plurality of bio-absorbable surgical clips is inserted into subcutaneous tissue and locked by the applicator.

7. The tissue closure clip dispenser system to dispense, bend, insert, and lock a plurality of bio-absorbable surgical clips for the rapid closure of a deep tissue wound of claim 1, wherein the first distal end of each of the plurality of bio-absorbable surgical clips further comprises a sharp end with a hook, and wherein the second distal end of each of the plurality of bio-absorbable surgical clips further comprises a catch end to receive the sharp end with the hook as the applicator locks each of the plurality of bio-absorbable surgical clips advanced between the sharp end with the hook and the catch end.

8. The tissue closure clip dispenser system to dispense, bend, insert, and lock a plurality of bio-absorbable surgical clips for the rapid closure of a deep tissue wound of claim 1, wherein the applicator to receive a dispensed bio-absorbable surgical clip from the dispenser further comprises:
a plurality of proximal handle levers to be held by an operator to operate the tissue closure clip dispenser system;
a fulcrum about which the proximal handle levers pivot while in use by the operator dispensing the plurality of bio-absorbable surgical clips; and
a set of distal ends having anvils to receive and hold each of the plurality of bio-absorbable surgical clips from the magazine and the dispenser in opposing surfaces of the distal ends and to subsequently bend each of the plurality of bio-absorbable surgical clips into a closed position.

9. The tissue closure clip dispenser system to dispense, bend, insert, and lock a plurality of bio-absorbable surgical clips for the rapid closure of a deep tissue wound of claim 1, wherein each of the plurality of bio-absorbable surgical clips comprises more than one color to aid viewing in a surgical repair.

10. The tissue closure clip dispenser system to dispense, bend, insert, and lock a plurality of bio-absorbable surgical clips for the rapid closure of a deep tissue wound of claim 1, wherein the plurality of bio-absorbable surgical clips are configured and utilized to facilitate the closure of fat on either side of an incision in advance of a cutaneous closure.

11. The tissue closure clip dispenser system to dispense, bend, insert, and lock a plurality of bio-absorbable surgical clips for the rapid closure of a deep tissue wound of claim 1, wherein each of the plurality of bio-absorbable surgical clips further comprises radio-opaque markers embedded within to aid in postoperative visualization and radiation therapy.

12. The tissue closure clip dispenser system to dispense, bend, insert, and lock a plurality of bio-absorbable surgical clips for the rapid closure of a deep tissue wound of claim 1, wherein each of the plurality of bio-absorbable surgical clips further comprises antibiotic and time-release pain medication embedded within.

13. The tissue closure clip dispenser system to dispense, bend, insert, and lock a plurality of bio-absorbable surgical clips for the rapid closure of a deep tissue wound of claim 8, wherein the set of distal ends having anvils to receive and hold each of the plurality of bio-absorbable surgical clips from the magazine and the dispenser in opposing surfaces of the distal ends and to subsequently bend each of the plurality of bio-absorbable surgical clips into a closed position is further comprised of grooves to guide each of the plurality of bio-absorbable surgical clips into tissue.

14. The tissue closure clip dispenser system to dispense, bend, insert, and lock a plurality of bio-absorbable surgical clips for the rapid closure of a deep tissue wound of claim 1, further comprising:
a central pointer, integrally formed in the magazine or the applicator of the tissue closure clip dispenser system, highlighting where the midline of the closed surgical wound will be once each of the plurality of bio-absorbable surgical clips is closed, thereby aiding a surgeon in assessing alignment and depth of surgical clip penetration.

15. The tissue closure clip dispenser system to dispense, bend, insert, and lock a plurality of bio-absorbable surgical clips for the rapid closure of a deep tissue wound of claim 1, wherein the magazine further comprises a radio-opaque marker embedded within.

16. The tissue closure clip dispenser system to dispense, bend, insert, and lock a plurality of bio-absorbable surgical clips for the rapid closure of a deep tissue wound of claim 1, wherein the magazine further comprises a light-emitting diode (LED) locating at an end of the magazine facing a surgical site, thereby illuminating the surgical site.

17. The tissue closure clip dispenser system to dispense, bend, insert, and lock a plurality of bio-absorbable surgical clips for the rapid closure of a deep tissue wound of claim 1, wherein each of the plurality of bio-absorbable surgical clips is comprised of one or more colors for conspicuous viewing during a tissue repair procedure.

18. The tissue closure clip dispenser system to dispense, bend, insert, and lock a plurality of bio-absorbable surgical clips for the rapid closure of a deep tissue wound of claim 1, wherein each of the plurality of bio-absorbable surgical clips is configured for a predetermined rate of resorption within a body, thereby accommodating surgical repairs requiring varied levels of structural integrity and longer periods of recuperation.

19. A method for loading a plurality of bio-absorbable surgical clips into a tissue closure clip dispenser system, the method comprising:
configuring a tissue closure clip dispenser system to comprise:
a plurality of bio-absorbable surgical clips, each bio-absorbable surgical clip formed as a unitary structure having a flexible elongated body with a first distal end and a second distal end, wherein the first distal end and the second distal end comprise a means for interlocking;
a magazine configured to contain the plurality of bio-absorbable surgical clips before any of the plurality of bio-absorbable surgical clips is dispensed in a sterile field;
a dispenser into which the magazine of the plurality of bio-absorbable surgical clips is loaded without breaking the sterile field, the dispenser configured to rapidly dispense the plurality of bio-absorbable surgical clips; and
an applicator to receive a dispensed bio-absorbable surgical clip from the dispenser, to rapidly bend, insert, and lock the dispensed bio-absorbable surgical clip into subcutaneous tissue, and to thereafter automatically receive a next dispensed bio-absorbable surgical clip from the dispenser;
loading the plurality of bio-absorbable surgical clips into the magazine;
placing the magazine of the plurality of bio-absorbable surgical clips into the dispenser;
installing the dispenser containing the magazine of the plurality of bio-absorbable surgical clips, in a substantially flush manner, to the applicator; and
replacing the magazine of the plurality of bio-absorbable surgical clips as needed during a closure of a deep tissue wound.

20. A method for rapidly dispensing a plurality of bio-absorbable surgical clips into subcutaneous tissue and for rapidly closing a deep tissue wound, the method comprising:
configuring a tissue closure clip dispenser system to comprise:
a plurality of bio-absorbable surgical clips, each bio-absorbable surgical clip formed as a unitary structure having a flexible elongated body with a first distal end and a second distal end, wherein the first distal end and the second distal end comprise a means for interlocking;
a magazine configured to contain the plurality of bio-absorbable surgical clips before any of the plurality of bio-absorbable surgical clips is dispensed in a sterile field;
a dispenser into which the magazine of the plurality of bio-absorbable surgical clips is loaded without breaking the sterile field, the dispenser configured to rapidly dispense the plurality of bio-absorbable surgical clips; and
an applicator comprising a plurality of proximal handle levers, a fulcrum, and a set of distal ends having anvils, the applicator to receive a dispensed bio-absorbable surgical clip from the dispenser, to rapidly bend, insert, and lock the dispensed bio-absorbable surgical clip into subcutaneous tissue, and to thereafter automatically receive a next dispensed bio-absorbable surgical clip from the dispenser;
loading the tissue closure clip dispenser system with the plurality of bio-absorbable surgical clips;
inserting the distal ends of the applicator into a tissue area to be closed;
pressing the proximal handle levers together to engage one of the plurality of bio-absorbable surgical clips into a closed position;
releasing the closed bio-absorbable surgical clip from the applicator;
dispensing additional bio-absorbable surgical clips as required by the tissue area being treated; and
retracting the distal ends of the applicator from the tissue area.
